(19) United States
(12) Patent Application Publication
(10) Pub. No.: US 2010/0185168 A1
(43) Pub. Date: Jul. 22, 2010

(54) URINATION DEVICE FOR FEMALE SCUBA DIVERS

(76) Inventors: Heleen Graauw, Houten (NL); Sander Jansson, Houten (NL); Laura J. Hanson, Seattle, WA (US)

Correspondence Address: FISH & RICHARDSON P.C., PO BOX 1022, MINNEAPOLIS, MN, 55440-1022, US

(21) Appl. No.: 12/355,302

(22) Filed: Jan. 16, 2009

(51) Int. Cl. A61F 5/455

(52) U.S. Cl. 604/347

(57) ABSTRACT

External urinary devices that can allow female scuba divers to utilize traditional overboard discharge (P-valve) systems.

### TECHNICAL FIELD

This document relates to an external urinary device that can allow female divers to utilize traditional overboard discharge (P-valve) systems.

### BACKGROUND

Scuba divers (e.g., technical divers) sometimes spend extended lengths of time underwater. It is important to be adequately hydrated while diving to avoid decompression and DCS. With adequate hydration, however, divers often need to urinate while they are in the water. Male divers can use external catheters and valves known in the art, but female divers traditionally have either had to rely on less desirable alternatives (e.g., using diapers or internal catheters, artificially limiting urine output by restricting hydration or increasing sodium intake), or wait until they are back on dry land to relieve themselves.

### SUMMARY

This document is based on the development of a device that allows female divers (e.g., female technical divers) to use a P-valve while diving in a drysuit, eliminating the need for diapers, internal catheters, and artificial limitation of urine output. The devices provided herein can permit females to execute long dives, multiple dives in a single day, and deep dives requiring extended bottom times, while wearing a drysuit without relying on diapers. The devices also can prolong the life of drysuit undergarments, and can keep female divers warmer under water than they would be if they had to wear a diaper under their drysuit. In addition, the devices provided herein can be used in any situation where conventional urination methods are not possible or not ideal, such as for patients (e.g., Amyotrophic lateral sclerosis (ALS) patients) who are not bedridden but who have difficulty using a toilet or controlling urination.

In one aspect, this document features an external urinary device for use by a female, the article comprising: (a) a reservoir surrounded by a flexible lip; and (b) a hollow, open-ended tube, wherein the tube comprises a proximal end connected to the reservoir and a lumen in fluid communication with the reservoir, wherein the article is adapted for reversible attachment of the lip to a female human body such that the reservoir is positioned over the urethral opening. The device can comprise a soft flexible polymer (e.g., silicone). The lip surrounding the reservoir can be essentially oval in shape. The lip can be about five inches to about seven inches long and about 2 to about 4 inches wide. The lip can be about 0.05 to about 0.075 inches thick. The tube can be about three inches to about five inches long. The tube can comprise a distal end, and wherein the device further comprises a fitting at the distal end and an extension tube attached to the fitting.

In another aspect, this document features a method of preparing an external urinary device for use by a female in scuba diving, the method comprising: (a) providing an external device comprising a reservoir surrounded by a flexible lip, and a hollow, open-ended tube having a distal end and a proximal end connected to the reservoir and a lumen in fluid communication with the reservoir; (b) applying an adhesive material to the flexible lip; (c) attaching the flexible lip with the applied adhesive material to a female human body such that the reservoir is positioned over the urethral opening; and (d) applying the distal end of the open-ended tube to a one-way valve that is adapted associated with a scuba suit to allow fluid from the open-ended tube to exit through the one-way valve but that does not allow dive water to enter the one-way valve and go in to the open-ended tube. The device can comprise a soft flexible polymer (e.g., silicone). The lip surrounding the reservoir can be essentially oval in shape. The lip can be about five inches to about seven inches long and about 2 to about 4 inches wide. The lip can be about 0.05 to about 0.075 inches thick. The tube can be about three inches to about five inches long. The tube can comprise a distal end, and wherein the device further comprises a fitting at the distal end and an extension tube attached to the fitting.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of the top side of a urination device as provided herein.

FIG. 2 is a perspective view of the underside of the urination device as provided herein and shown in FIG. 1.

FIG. 3 is a side view of the urination device as provided herein and shown in FIGS. 1 and 2.

FIG. 4 is an underside view of the urination device as provided herein and shown in FIGS. 1-3.

FIG. 5 is an end view of the urination device as provided herein and shown in FIGS. 1-4.

FIG. 6 is a flow chart illustrating a method of preparing and applying a device such as the urination device shown in the preceding figures for use by a female in scuba diving.

Like reference symbols in the various drawings indicate like elements.

### DETAILED DESCRIPTION

This document is based on the development of a device that makes it possible for female divers (e.g., female technical divers) to use a P-valve while diving in a drysuit, eliminating the need for diapers. The device in general can be made from a soft, flexible polymer (e.g., silicone, latex, polyurethane, or polyvinylchloride), and can have a reservoir, an output tube, and a flange that can be adhered to the skin with, for example, a medical adhesive. The device can be configured and sized such that it can be positioned on the skin between the perineum and the pubic bone to provide a proper seal and comfortable urination without leakage.

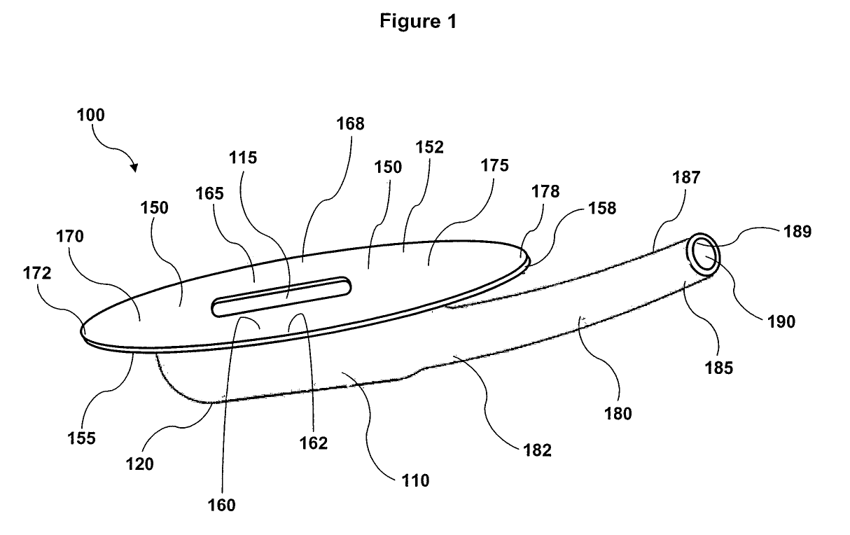

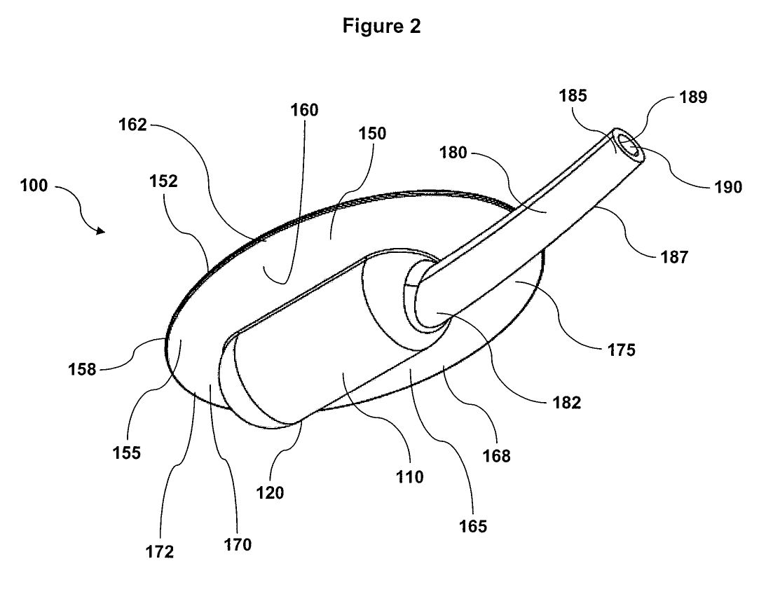

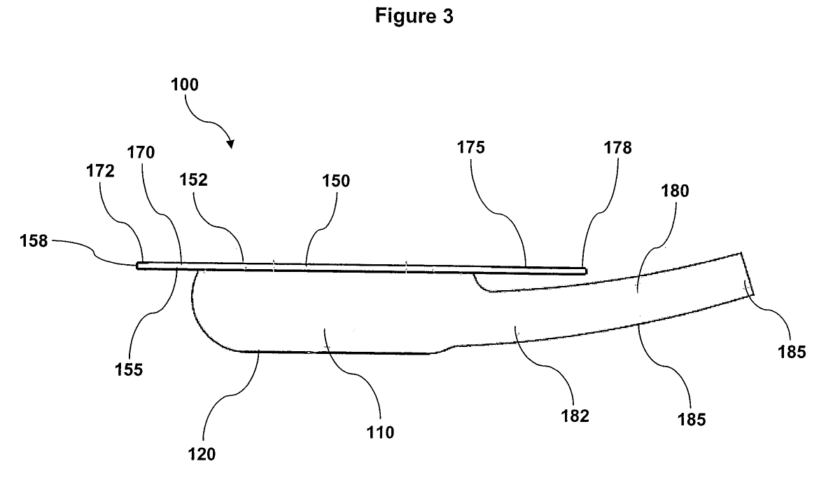

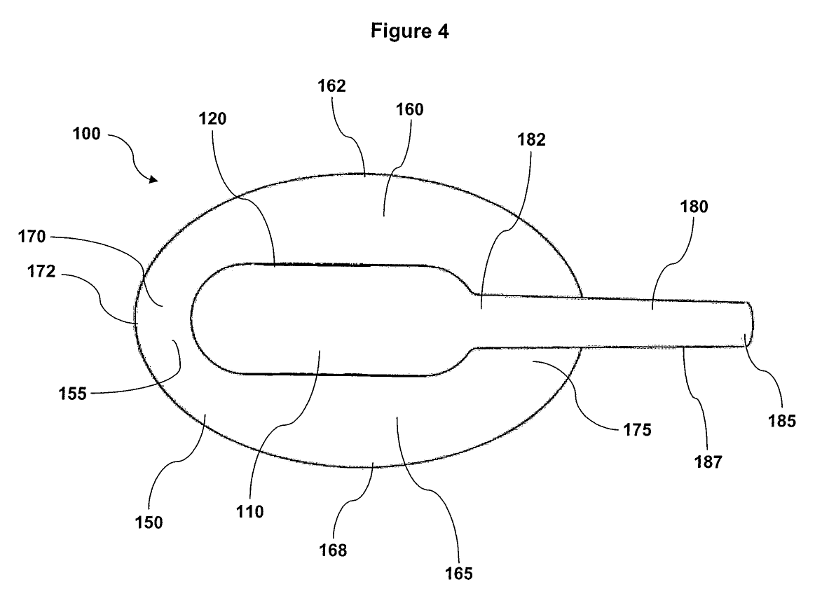

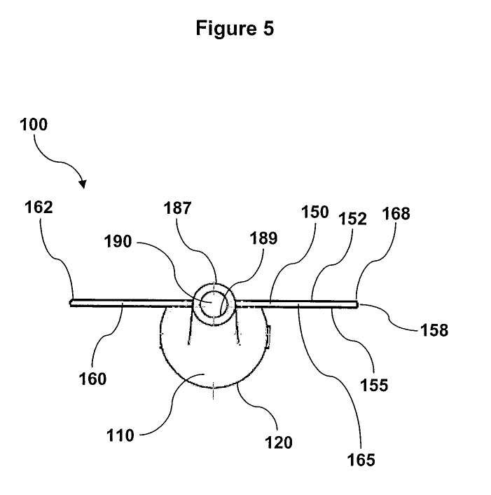

In general, the devices provided herein include a reservoir for collecting urine, a tube through which urine can exit the device, and a lip or flange surrounding the reservoir, which can be firmly and removably attached to a female human body around the urethral opening. As shown in FIGS. 1-5, for example, device 100 can have reservoir 110 surrounded by flange 150. Reservoir 110 can have inner surface 115 and outer surface 120. Flange 150 can have top surface 152, bottom surface 155 opposite top surface 152, and exterior edge 158 extending between top and bottom surfaces 152 and 155. Top surface 152 of flange 150 can be continuous with inner surface 115 of reservoir 110, and bottom surface 155 of flange 150 can be continuous with outer surface 120 of reservoir 110.

Flange 150 can have a shape and dimensions that permit device 100 to be positioned securely and reversibly over the female anatomy, such that urine can pass from the urethral opening into reservoir 110. The shape of flange 150 typically is elongated, with a length sufficient for attachment to the skin from the perineum to the top of the pubic bone, and a width sufficient for attachment over the external labia. As depicted in FIGS. 1-5, for example, flange 150 can have a substantially oval shape. In some cases, flange 150 can have a more rectangular shape (e.g., a rectangle with rounded corners). Flange 150 can have right side portion 160 with right side edge 162, left side portion 165 with left side edge 168, dorsal end portion 170 with dorsal end edge 172, and ventral end portion 175 with ventral end edge 178. The length of flange 150 between dorsal end edge 172 and ventral end edge 178 can be from about 4 inches to about 8 inches (e.g., about 4, about 4.25, about 4.5, about 4.75, about 5, about 5.25, about 5.5, about 5.75, about 6, about 6.25, about 6.5, about 6.75, about 7, about 7.25, about 7.5, about 7.75, or about 8 inches), or any dimension or range there between. The width of flange 150 between right side edge 162 and left side edge 168 can be from about 2 inches to about 4 inches (e.g., about 2, about 2.25, about 2.5, about 2.75, about 3, about 3.25, about 3.5, about 3.75, or about 4 inches), or any dimension or range there between.

Flange 150 can have a thickness that is provides sufficient strength for multiple attachments and removals of device 100, but that also provides sufficient flexibility for comfortable wear by a user. For example, the thickness of flange 150 can be from about 0.025 inch to about 0.12 inch (e.g., about 0.025, about 0.03, about 0.04, about 0.05, about 0.06, about 0.07, about 0.075, about 0.08, about 0.09, about 0.1, about 0.11, or about 0.12 inch), or any dimension or range there between. Further, the overall height of device 100 can be between about 1 inch and about 2 inches (e.g., about 1, about 1.25, about 1.5, about 1.75, or about 2 inches), or any dimension or range there between.

Reservoir 110 can be centrally located with respect to flange 150, or can be acentrally located. For example, as depicted in FIGS. 1-4, reservoir 110 can be located essentially equidistant between right side edge 162 and left side edge 168, and closer to dorsal end edge 172 than ventral end edge 178. An acentrally located reservoir can be particularly useful, such that when dorsal end portion 170 of flange 150 is attached to the perineum, reservoir 110 is positioned over the urethral opening, and ventral end portion is attached between the clitoral hood and the end of the top of the pubic bone.

Device 100 can further include hollow tube 180 that can have proximal portion 182, distal end 185, outer surface 187, and inner surface 189 that defines lumen 190. Outer surface 187 of tube 180 can extend from outer surface 120 of reservoir 110, and lumen 190 of tube 180 can be in fluid communication with the interior of reservoir 110. In some embodiments (e.g., as depicted in FIGS. 1-4), tube 180 can extend from a portion of reservoir 110 that is adjacent to ventral end portion 175 of flange 150, such that tube 180 protrudes over ventral end portion 175 and beyond ventral end edge 178. Tube 180 can have a length of, for example, about 2.5 inches to about 5.5 inches in length (e.g., about 2.5, about 2.75, about 3, about 3.25, about 3.5, about 3.75, about 4, about 4.25, about 4.5, about 4.75, about 5, about 5.25, or about 5.5 inches), or any dimension or range there between.

Distal end 185 of tube 180 can have a straight or beveled opening, or can have a particular configuration for attachment to another piece of apparatus (e.g., a tube connected to a P-valve in a drysuit, or an extension tube that in turn can be connected to P-valve tubing). In some cases, distal end 185 can have a barbed fitting to facilitate a compression fit with a further piece of tubing. In some cases, distal end 185 also can be fitted with a quick disconnect plug, which can facilitate connection tube 180 with P-valve tubing, as well as disconnection of tube 180 from P-valve tubing. In some embodiments, distal end 185 can be configured for connection to an extension tube (e.g., a piece of NORPRENE® or rubber tubing) via a compression fit, for example, and the extension tube in turn can be configured for connection to a P-valve hose via a quick disconnect fitting, for example.

To use a device as described herein, one can apply an adhesive to top surface 152 of flange 150. The adhesive can be a paste, a spray, a tape, or a combination thereof. For example, a paste or spray adhesive (e.g., a medical adhesive spray or paste for prosthetic and ostomy devices, as available from Hollister Incorporated, Libertyville, Ill.) can be spread over top surface 152. An even, continuous coating of adhesive over top surface 152 can be particularly useful to prevent leakage, for example. In some cases, one or more pieces of tape can be attached to bottom surface 155 of flange 150, such that the ends of the tape pieces can hold device 100 against the skin. In some embodiments, double sided tape can be used.

After coating with adhesive and/or placement of tape, device 100 can be attached to the skin. Dorsal end portion 170 can be positioned on the perineum just adjacent to, but not covering, the anus. Right and left side portions 160 and 165 can be positioned on the right and left outer labia. Ventral end portion 175 can be attached to the skin from the clitoral hood to the pubic bone. In some cases, device 100 can be attached to the skin with paste or spray adhesive, and tape can be added to secure the attachment. Using tape to secure dorsal and ventral end portions 170 and 175 can be particularly useful for preventing leaks, for example. Further, attaching device 100 to skin that is dry, at least partially shaven, and free of residue from razor moisturizing strips, for example, also can facilitate achieving a good seal and preventing leaks.

When device 100 is positioned on a user as described above, tube 180 will extend forward (i.e., ventrally) from device 100. Distal end 185 of tube 180 can be passed through an opening in a drysuit undergarment and connected to a further piece of tubing (e.g., extension tubing or P-valve tubing). In some embodiments, tube 180 can be connected to extension tubing (e.g., via a compression fit) prior to placement of device 100 on the skin. An extension tube can be, for example, about 2.5 inches to about 5.5 inches in length (e.g., about 2.5, about 2.75, about 3, about 3.25, about 3.5, about 3.75, about 4, about 4.25, about 4.5, about 4.75, about 5, about 5.25, or about 5.5 inches in length) or any dimension or range there between.

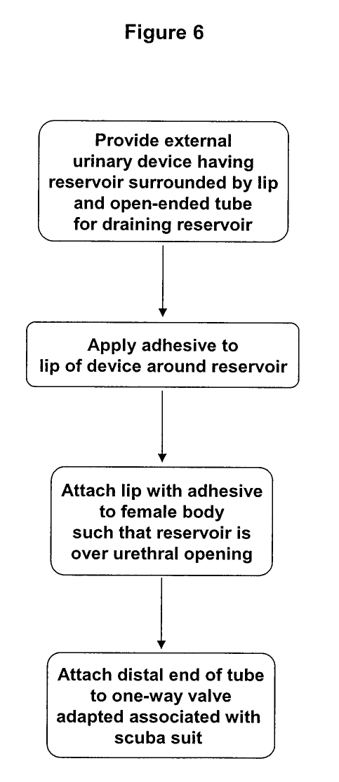

In summary, and referring now to FIG. 6, there is shown a flow chart that illustrates a method of preparing and applying a device such as the urination device described herein and depicted in FIGS. 1-5 for use by a female in scuba diving.

In use, a female can urinate using device 100 such that urine passes through reservoir 110, tube 180, and any extension tubing and/or P-valve tubing to which tube 180 is connected. Device 100 can be particularly useful when a user is in an upright (e.g., standing or sitting) or prone position (e.g., as when scuba diving). It is noted that the devices described herein also can be useful for females who, for medical reasons, have difficulty using a toilet or controlling urination. For example, a female having a disease such as ALS may benefit from using a device as described herein, which is less invasive than an internal catheter, and which may be more sanitary, comfortable, and convenient than a diaper. In such instances, tube 180 of device 100 (or an extension tube attached to tube 180) can be connected to a catheter bag, or can be drained directly into a toilet.

Device 100 can be detached from the skin after use, and the adhesive can be removed by, for example, allowing the adhesive to dry and then peeling it off device 100 and/or the skin. Residual adhesive can be removed using methods and materials known in the art. For example, baby oil or soap and water can be used to remove adhesive from the skin, while alcohol, acetone, vinegar, or a commercially available remover such as REMOVE™ (Smith & Nephew, London, UK), ALLKARE® (ConvaTec Professional Services, Skillman, N.J.), or DETACHOL (Ferndale Laboratories, Inc., Ferndale, Mich.). Device 100 can be rinsed, washed (e.g., with soapy water), disinfected (e.g., with isopropyl alcohol), and/or dried after the adhesive is removed, such that it will be ready for its next use. The material from which device 100 is made can affect how it is cleaned after use. For example, if device 100 contains latex, residual adhesive can be removed with alcohol or acetone, and device 100 can be rinsed and then disinfected with isopropyl alcohol. If device 100 contains silicone, a commercially available adhesive remover can be used, and device 100 can be cleaned with warm, soapy water. In such embodiments, device 100 can be used numerous times.

In some embodiments, a device as provided herein can be designed to be disposable after one or several uses. For example, a device that is manufactured to be disposable can be made from a material such as polyvinyl chloride or polyurethane. Further, the device can be manufactured using a number of methods known in the art, including using hand molds or injection molding.

This document also provides articles of manufacture that can include one or more devices as described herein. In some embodiments, an article of manufacture can include one or more devices as described herein in combination with one or more pieces of extension tubing, one or more fittings (e.g., a barbed fitting and/or a quick disconnect fitting), an adhesive (e.g., a paste, spray, or tape adhesive), and/or an adhesive remover. In some cases, a device as described herein can have a flange that is pre-coated with an adhesive. For example, a flange can be coated with an adhesive and covered with a releasable sheet, which can be peeled off the device prior to application to the skin.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An external urinary device for use by a female, the article comprising:
(a) a reservoir surrounded by a flexible lip; and
(b) a hollow, open-ended tube, wherein the tube comprises a proximal end connected to the reservoir and a lumen in fluid communication with the reservoir,
wherein the article is adapted for reversible attachment of the lip to a female human body such that the reservoir is positioned over the urethral opening.

2. The device of claim 1, wherein the device comprises a soft flexible polymer.

3. The device of claim 1, wherein the device comprises silicone.

4. The device of claim 1, wherein the lip surrounding the reservoir is essentially oval in shape.

5. The device of claim 1, wherein the lip is about five inches to about seven inches long and about 2 to about 4 inches wide.

6. The device of claim 1, wherein the lip is about 0.05 to about 0.075 inches thick.

7. The device of claim 1, wherein the tube is about three inches to about five inches long.

8. The device of claim 1, wherein the tube comprises a distal end, and wherein the device further comprises a fitting at the distal end and an extension tube attached to the fitting.

9. A method of preparing an external urinary device for use by a female in scuba diving, the method comprising:
(a) providing an external device comprising a reservoir surrounded by a flexible lip, and a hollow, open-ended tube having a distal end and a proximal end connected to the reservoir and a lumen in fluid communication with the reservoir;
(b) applying an adhesive material to the flexible lip;
(c) attaching the flexible lip with the applied adhesive material to a female human body such that the reservoir is positioned over the urethral opening; and
(d) applying the distal end of the open-ended tube to a one-way valve that is adapted associated with a scuba suit to allow fluid from the open-ended tube to exit through the one-way valve but that does not allow dive water to enter the one-way valve and go in to the open-ended tube.

10. The method of claim 9, wherein the device comprises a soft flexible polymer.

11. The method of claim 9, wherein the device comprises silicone.

12. The method of claim 9, wherein the lip surrounding the reservoir is essentially oval in shape.

13. The method of claim 9, wherein the lip is about five inches to about seven inches long and about 2 to about 4 inches wide.

14. The method of claim 9, wherein the lip is about 0.05 to about 0.075 inches thick.

15. The method of claim 9, wherein the tube is about three inches to about five inches long.

16. The method of claim 9, wherein the tube comprises a distal end, and wherein the device further comprises a fitting at the distal end and an extension tube attached to the fitting.
